(19) United States
(12) Patent Application Publication
(10) Pub. No.: US 2015/0305770 A1
(43) Pub. Date: Oct. 29, 2015

(54) SYSTEM AND METHOD OF IMAGE GUIDED PERICARDIAL PROCEDURES INCLUDING PERICARDIOSCOPY, PERICARDIAL ABLATION, PERICARDIAL MATERIAL DELIVERY, PERICARDIAL TISSUE GRASPING AND MANIPULATION, PERICARDIAL LEAD PLACEMENT, AND PERICARDIAL SURGICAL FASTENER PLACEMENT

(71) Applicant: Epicardial Frontiers, LLC, Wexford, PA (US)

(72) Inventors: Brian Fill, Pittsburgh, PA (US); Mark J. Gartner, Pittsburgh, PA (US); Prahlad Menon Gopalakrishna, Pittsburgh, PA (US); Ganesh Mani, Pittsburgh, PA (US); Daniel Ludwig, Pittsburgh, PA (US)

(21) Appl. No.: 14/645,809

(22) Filed: Mar. 12, 2015

Related U.S. Application Data
(60) Provisional application No. 61/951,528, filed on Mar. 12, 2014.

(51) Int. Cl. A61B 17/34; A61B 19/00; A61B 6/03; A61B 5/06; A61B 17/02

(52) U.S. Cl. CPC A61B 17/3403; A61B 5/061; A61B 17/0218; A61B 6/032; A61B 19/50; A61B 2017/0237; A61B 2019/507

(57) ABSTRACT

A system and method of performing percutaneous image guided pericardial procedures is disclosed. The method comprises the steps of: providing a tool tracking system having a display for visualization of virtual representations of tracked tools which is configured to have a field of operation encompassing percutaneous pericardial space access and procedures; providing a patient specific three dimensional model of the patient for use in percutaneous pericardial space procedures that is registered with the tool tracking system for simultaneous display of the model and the virtual representation of the tracked tools on the system display; percutaneously inserting at least one tracked tool for the tool tracking system into the pericardial space; and simultaneously visualizing the virtual representation of the tracked tool and the patient specific model on the system display at least through a portion of a pericardial procedure.

### RELATED APPLICATIONS

This application claims priority to U.S. provisional patent application Ser. No. 61/951,528 filed Mar. 12, 2014, entitled “Method and Apparatus for Access of the Pericardial Space.”

### BACKGROUND OF THE INVENTION

1. Field of the Invention

The present invention relates to technologies to facilitate access to the pericardial space and to perform procedures on the heart from the pericardial space, more specifically the present invention relates to pericardial space access tools that minimize potential myocardial damage via cardiac perforation or coronary laceration.

2. Background Information

The pericardial space, also called the pericardial cavity or pericardial sac, is an opportune staging site for a multitude of existing and emerging therapeutic technologies. Some of these are discussed in U.S. Pat. No. 8,475,468 entitled Method and Apparatus for Providing Intra-pericardial Access.” See also U.S. Pat. No. 8,246,539 entitled “Pericardium Management Method for Intra-pericardial Surgical Procedures”; U.S. Pat. No. 8,147,413 entitled “Image Guided Catheter having Deployable Balloons and Pericardial Access Procedure”; U.S. Pat. No. 8,075,532 entitled “Devices, Systems, and Methods for Pericardial Access”; U.S. Pat. No. 7,309,328 entitled “Method and Apparatus for Pericardial Access”; U.S. Pat. No. 6,918,890 entitled “Direct Pericardial Access Device and Method”; U.S. Pat. No. 6,666,844 entitled “Method and Apparatus for Accessing the Pericardial Space”; U.S. Pat. No. 6,613,062 entitled “Method and Apparatus for Providing Intra-Pericardial Access”; U.S. Pat. No. 6,592,553 entitled “Direct Pericardial Access Device and Method”; U.S. Pat. No. 6,423,051 entitled “Method and Apparatus for Pericardial Access”; U.S. Pat. No. 6,162,195 entitled “Method and Apparatus for Accessing the Pericardial Space”; U.S. Pat. No. 6,156,009 entitled “Apparatus for Accessing the Pericardial Space”; U.S. Pat. No. 5,972,013 entitled “Direct Pericardial Access Device with Deflecting Mechanism and Method”; and U.S. Pat. No. 5,931,810 entitled “Method for Accessing the Pericardial Space.” These patents are incorporated herein by reference.

There are many different cardiac drugs that have been delivered safely into the pericardial space. For example note “Amiodarone instilled into the canine pericardial sac migrates transmurally to produce electrophysiologic effects and suppress atrial fibrillation.” Ayers G M, Rho T H, Ben-David J, Besch H R, Jr, Zipes D P., J Cardiovasc Electrophysiol. 1996;7:713-721. The beneficial effects of nitric oxide and nitroprusside in ischemic animal model have been studied, “Localized administration of sodium nitroprusside enhances its protection against platelet aggregation in stenosed and injured coronary arteries” Willerson J T, Igo S R, Yao S K, Ober J C, Macris M P, Ferguson J J Tex Heart Inst J. 1996; 23(1):1-8. Further myosin antibody loaded with anti-arrhythmic agents has been delivered via the pericardial space to demonstrate the efficacy. Iontophoresis has been proposed to enhance drug efficacy via the pericardial space in “Iontophoretic transmyocardial drug delivery. A novel approach to anti-arrhythmic drug therapy.”Avitall B, Hare J, Zander G, Bockoff C, Tchou P, Jazayeri M, Akhtar M, Circulation. 1992 April; 85(4):1582-93. See also “Persistent primary coronary dilation induced by transatrial delivery of nitroglycerin into the pericardial space: a novel approach for local cardiac drug delivery.” Waxman S, Moreno R, Rowe K A, Verrier R L, J Am Coll Cardiol. 1999 June; 33(7):2073-7. See also “Angiogenic therapy of acute myocardial infarction by intrapericardial injection of basic fibroblast growth factor and heparin sulfate: an experimental study.” Uchida Y, Yanagisawa-Miwa A, Nakamura F, Yamada K, Tomaru T, Kimura K, Morita T Am Heart J. 1995 December; 130(6):1182-8. See also U.S. Pat. No. 8,123,716 entitled “Pericardial Delivery of Treatment”; U.S. Pat. No. 6,692,458 entitled “Intra-Pericardial Drug Delivery Device with Multiple Balloons and Method for Angiogenesis”; U.S. Pat. No. 6,206,004 entitled “Treatment Method via the Pericardial Space.”

The pericardial space is likely to be an opportune staging site for myocardial gene or stem cell injections. This is evidenced in: “Review Biological therapies for cardiac arrhythmias: can genes and cells replace drugs and devices?”Cho H C, Marbán E Circ Res. 2010 Mar. 5; 106(4):674-85; see also “Efficient in vivo catheter-based pericardial gene transfer mediated by adenoviral vectors.” March K L, Woody M, Mehdi K, Zipes D P, Brantly M, Trapnell B C, Clin Cardiol. 1999 January; 22(1 Suppl 1):123-9; see also “Epicardial border zone overexpression of skeletal muscle sodium channel SkM1 normalizes activation, preserves conduction, and suppresses ventricular arrhythmia: an in silico, in vivo, in vitro study”, Lau D H, Clausen C, Sosunov E A, Shlapakova I N, Anyukhovsky E P, Danilo P Jr, Rosen T S, Kelly C, Duffy H S, Szabolcs M J, Chen M, Robinson R B, Lu J, Kumari S, Cohen I S, Rosen M R, Circulation. 2009 Jan. 6; 119(1):19-27.). See U.S. Pat. No. 5,797,870 entitled “Pericardial Delivery of Therapeutic and Diagnostic Agents”.

The pericardial space is an opportune staging site for arrhythmia treatments such as epicardial ablation, or ablation of cells on the outside the heart muscle. In order to perform ablation on the outside of the heart, it is first necessary to find a route to the outside of the heart without requiring surgery. The most direct and safest route to the space outside the heart is the region just under the breastbone at the bottom of the rib cage. A needle enters the pericardial space and then a guidewire is inserted into the pericardial space to allow the ablation catheter to be safely inserted into the pericardial space. A guide tube may replace the guidewire to accommodate the ablation catheter. Once the ablation catheter is positioned in the pericardial space the exact site of the heart rhythm problem may be identified from the outside of the heart and it treated with ablation. See also U.S. Pat. No. 8,287,532 entitled “Epicardial Mapping and Ablation Catheter”; U.S. Pat. No. 7,794,454 entitled “Method and Device for Epicardial Ablation”; U.S. Pat. No. 7,625,396 entitled “Method and Device for Epicardial Ablation”; U.S. Pat. No. 7,041,099 entitled “Intraoperative Endocardial and Epicardial Ablation Probe”; U.S. Pat. No. 6,960,205 entitled “Suction Stabilized Epicardial Ablation Devices”; U.S. Pat. No. 6,887,238 entitled “Suction Stabilized Epicardial Ablation Devices”; U.S. Pat. No. 6,558,382 entitled “Suction Stabilized Epicardial Ablation Devices”; U.S. Pat. No. 6,540,742 entitled “Intraoperative Endocardial and Epicardial Ablation Probe”; U.S. Pat. No. 6,514,250 entitled “Suction Stabilized Epicardial Ablation Devices”; and U.S. Pat. No. 6,237,605 entitled “Methods of Epicardial Ablation.” These patents are incorporated herein by reference.

The pericardial space is an opportune staging site for lead placement, see “A transatrial pericardial access: lead placement as proof of concept”, G. S. Kassab , M. Svendsen , W. Combs , J. S. Choy , E. J. Berbari , J. A. Navia, American Journal of Physiology—Heart and Circulatory Physiology Published 1 Jan. 2010 Vol. 298 no. H287-H293. See also U.S. Pat. No. 8,036,757 entitled “Pacing Lead and Method for Pacing in the Pericardial Space”; U.S. Pat. No. 7,797,059 entitled “System and Method for Lead Placement in a Pericardial Space”; U.S. Pat. No. 7,272,448 directed to a “Medical Lead for Placement in the Pericardial Sac”; and U.S. Pat. No. 5,052,407, which patents are incorporated herein by reference.

The pericardial space is an opportune staging site for left atrial appendage (LAA) manipulations, as discussed in U.S. Pat. Nos. 8,007,504; 7,527,634 and 6,488,489 and U.S. Published Patent Application 2002-0099390 and 2012-0327204, and these patents and published patent applications are incorporated herein by reference. Also note the article “Percutaneous epicardial left atrial appendage closure: preliminary results of an electrogram guided approach.” Friedman P A, Asirvatham S J, Dalegrave C, Kinoshita M, Danielsen A J, Johnson S B, Hodge D O, Munger T M, Packer D L, Bruce C J, J Cardiovasc Electrophysiol. 2009 August; 20(8):908-15. The authors of the “A transatrial pericardial access: lead placement as proof of concept” noted that “a nonsurgical, percutaneous device that permits rapid and safe access into the pericardial space is highly desirable and would have significant potential for expanding cardiac diagnostics and therapies.” The present invention satisfies this identified need.

Clinically, the only current commercial nonsurgical means for accessing the pericardial space is the subxiphoid needle approach. Specifically conventional pericardial access is attempted using a commercial needle with a “Tuohy” shape, with puncture based on significant tactile pressure/feedback. More problematically, current pericardial access techniques are dominated by mind's eye work. This work is complicated by a lack of “feel” and visual obscuration based on serial dye injection. Further, the current Tuohy needle is poorly shaped for this purpose as an angulated bevel positions the leading edge of the needle well beyond the trailing edge. As a result of these and other complications, currently “dry” pericardial access is beyond the scope of many clinicians, x-ray-intensive, and associated with a significant rate of right ventricular perforation. For general background see “Percutaneous pericardiocentesis versus subxiphoid pericardiotomy in cardiac tamponade due to postoperative pericardial effusion.”, Susini G, Pepi M, Sisillo E, Bortone F, Salvi L, Barbier P, Fiorentini C., J Cardiothorac Vasc Anesth 7: 178-183, 1993; and “Subxiphoid pericardiocentesis guided by contrast two-dimensional echocardiography in cardiac tamponade: experience of 110 consecutive patients”. Vayre F, Lardoux H, Pezzano M, Bourdarias J P, Dubourg O., Eur J Echocardiogr 1: 66-71, 2000.

The known pericardial access needle may be guided, to a minimal extent, by fluoroscopy, which may cause undesirable radiation exposure to the operator who is positioned directly against the image intensifier. One proposed method to address these prior art difficulties includes use of a sheathed needle with a suction tip designed for grasping the pericardium and accessing the pericardial space using a transthoracic approach, while avoiding myocardial puncture. This device is advanced from a subxiphoid position into the mediastinum under fluoroscopic guidance and positioned onto the anterior outer surface of the pericardial sac. In diseased or dilated hearts, the pericardial space is significantly smaller than normal, and the risk of puncture of the right ventricle (RV) or other cardiac structures is more prominent.

Thus current commercial and proposed percutaneous pericardial access carries an associated risk of cardiac perforation or coronary laceration. Further pericardial space access only guided by fluoroscopic imaging offers only a limited two-dimensional silhouette visualization field of anatomy and tools.

Furthermore, in any given parietal pericardial territory being considered for puncture, there are regions of access which are safer than others. For example, anteroapical access would be safer if performed in a region where there was a fat pad overlying the contiguous right ventricular wall. In addition, epicardium-based procedures are increasingly geographic because the underlying pathology is increasingly well defined. Ventricular tachycardia (VT), for example, is supported by pathological tissue that is now routinely identified by preoperative (computed tomography, magnetic resonance, nuclear imaging) images. Advance knowledge of likely ablation target territory permits strategic pericardial puncture as to minimize the complexity of subsequent catheter manipulation.

With this background it remains clear that a nonsurgical, percutaneous apparatus and associated method that permits rapid and safe access into the pericardial space is highly desirable and would have significant potential for expanding cardiac diagnostics and therapies, and there remains a need for such a method and apparatus that is cost effective and easy for the medical professionals to implement.

### SUMMARY OF THE INVENTION

One aspect of the present invention provides an apparatus for accessing the pericardial space including a percutaneous pericardium access tool having a lumen sufficient for emitting dye visible on real time imaging through the lumen and wherein a leading edge of the needle of the percutaneous pericardium access tool is formed to minimize the likelihood of myocardial laceration or puncture. The apparatus of accessing the pericardial space according to invention may provide that the percutaneous pericardium access tool includes a needle at a distal end thereof, and wherein the needle of the percutaneous pericardium access tool is formed with a larger proximal diameter than the smaller distal diameter. The apparatus for accessing the pericardial space according to the present invention may further include a guide, such as a guidewire, configured to be inserted into the pericardial space through a pericardium opening formed by the percutaneous pericardium access tool.

One aspect of this invention is directed to an method of image guided access of the pericardial space via a pericardial space access port comprises the steps of: forming an opening in the pericardium of a patient; placing a percutaneous port into the opening in the pericardium, wherein the port is configured to receive tools there through into the pericardial space; providing a tool tracking system having a display for visualization of virtual representations of tracked tools which is configured to have a field of operation encompassing percutaneous pericardial space access; providing a patient specific three dimensional model of the patient for use in percutaneous pericardial space access that is registered with the tool tracking system for simultaneous display of the model and the virtual representation of the tracked tools on the system display; inserting at least one tracked tool for the tool tracking system into the pericardial space through the port; and simultaneously visualizing the virtual representation of the tracked tool and the patient specific model on the system display.

One aspect of this invention is directed to a method of accessing the pericardial space of a patient comprising the steps of: Forming a patient specific three dimensional model of the patient for use in percutaneous pericardial space access; Configuring a tool tracking system having a display for visualization of virtual representations of tracked tools to have a field of operation encompassing percutaneous pericardial space access; Registering the three dimensional model with the tool tracking system for simultaneous display of the patient specific three dimensional model and the virtual representations of tracked tools on the system display; Providing a percutaneous pericardium access tool with a tool tracking sensor for use with the tool tracking system and with a virtual tool representation for the display; Simultaneously displaying the virtual tool representation of the percutaneous pericardium access tool and the patient specific three dimensional model of the patient at least during movement of the percutaneous pericardium access tool toward the pericardium; and Creating an opening in the pericardium with the percutaneous pericardium access tool.

It is noted that, as used in this specification and the appended claims, the singular forms “a,” “an,” and “the” include plural referents unless expressly and unequivocally limited to one referent. The features that characterize the present invention are pointed out with particularity in the claims which are part of this disclosure. These and other features of the invention, its operating advantages and the specific objects obtained by its use will be more fully understood from the following detailed description and the operating examples.

The method of accessing the pericardial space according to the present invention may further include the step of inserting a guide into the pericardial space through the formed pericardium opening, and wherein the guide is in the form of a guidewire.

The method of accessing the pericardial space according to the present invention may further include the step of obtaining a medical scan of the patient and wherein the forming the patient specific three dimensional model is based upon the medical scan, wherein the medical scan is one of an MRI and a CT scan. The obtaining of a medical scan may include placing scan-able registration markers on the patient, and wherein the patient specific three dimensional model includes representation of the registration markers, and wherein the step of registering the three dimensional model with the tool tracking system includes placing the percutaneous pericardium access tool on a plurality of the registration markers and coordinating the known position of the percutaneous pericardium access tool as determined by the tool tracking system with model position of the associated representation of the registration marker.

The method of accessing the pericardial space according to the present invention may further include that the patient specific model details cardiac landmarks utilized during pericardial access to minimize risks associated with accidental myocardial puncture. Further, the cardiac landmarks utilized during pericardial access to minimize risks associated with accidental myocardial puncture may include areas with fatty deposits, and wherein these fatty areas are targeted as general areas to proceed with forming the opening in the pericardium in order to minimize the risks associated with accidental myocardial puncture.

The method of accessing the pericardial space according to the present invention may further include the step of utilizing real time imaging, such as fluoroscopy at least during the creating of the opening in the pericardium with the percutaneous pericardium access tool. Further, the percutaneous pericardium access tool may includes a lumen, and may further include the step of emitting dye visible on the real time imaging through the lumen.

One aspect of this invention is directed to an apparatus for accessing the pericardial space of a patient comprising: a tool tracking system having a display for visualization of virtual representations of tracked tools which is configured to have a field of operation encompassing percutaneous pericardial space access; a patient specific three dimensional model of the patient for use in percutaneous pericardial space access that is registered with the tool tracking system for simultaneous display of the model and the virtual representation of the tracked tools on the system display; and a percutaneous pericardium access tool with a tool tracking sensor for use with the tool tracking system and with a virtual tool representation for the display.

The apparatus of accessing the pericardial space according to the invention may provide that the patient specific three dimensional model is based upon a medical scan of the patient and wherein the medical scan includes placing scan-able registration markers on the patient, and wherein the patient specific three dimensional model includes representation of the registration markers. The patient specific model may detail cardiac landmarks utilized during pericardial access to minimize risks associated with accidental myocardial puncture, and wherein the cardiac landmarks utilized during pericardial access to minimize risks associated with accidental myocardial puncture include areas with fatty deposits, and wherein these fatty areas are targeted as general areas to proceed with forming the opening in the pericardium in order to minimize the risks associated with accidental myocardial puncture.

The apparatus of accessing the pericardial space according to invention may provide that the percutaneous pericardium access tool includes a lumen sufficient emitting dye visible on real time imaging through the lumen. The apparatus of accessing the pericardial space according to invention may provide that the percutaneous pericardium access tool includes a needle at a distal end thereof, and wherein the needle of the percutaneous pericardium access tool is formed with a larger proximal diameter than the smaller distal diameter, or wherein a leading edge of the needle of the percutaneous pericardium access tool is formed to minimize the likelihood of myocardial laceration or puncture.

The apparatus of accessing the pericardial space according to one aspect of the invention provides that a distal end of the percutaneous pericardium access tool includes a radio frequency ablation tool.

It is noted that, as used in this specification and the appended claims, the singular forms “a,” “an,” and “the” include plural referents unless expressly and unequivocally limited to one referent. The features that characterize the present invention are pointed out with particularity in the claims which are part of this disclosure. These and other features of the invention, its operating advantages and the specific objects obtained by its use will be more fully understood from the following detailed description and the operating examples.

One aspect of the present invention provides a method of accessing the pericardial space of a patient comprising the steps of: forming an opening in the pericardium of a patient; placing a guidewire to extend through the formed opening in the pericardium and into the pericardial space; dilating the formed opening in the pericardium; and placing a percutaneous port into the dilated opening in the pericardium, wherein the port is configured to receive tools there through into the pericardial space.

The method of accessing the pericardial space according to the invention may further include the step of deploying an anchor mechanism to maintain the percutaneous port within the dilated opening in the pericardium. The anchor mechanism includes an anchor cuff within the pericardial space and may further include a proximal supra-dermal anchor cuff.

The method of accessing the pericardial space according to the invention may provide that the percutaneous port includes multiple distinct lumens and may further including the step of insufflating the pericardial space via the percutaneous port.

One aspect of the present invention provides an image guided pericardial access port assembly for accessing the pericardial space of a patient comprising: a tool tracking system having a display for visualization of virtual representations of tracked tools which is configured to have a field of operation encompassing percutaneous pericardial space access; a patient specific three dimensional model of the patient for use in percutaneous pericardial space access that is registered with the tool tracking system for simultaneous display of the model and the virtual representation of the tracked tools on the system display; a percutaneous port configured to extend through an opening in the pericardium, wherein the port is configured to receive tools there through into the pericardial space; and at least one tracked tool for the tool tracking system configured to be received through the percutaneous port.

The image guided pericardial access port assembly according the invention may include that the percutaneous port further includes an anchor mechanism to maintain the percutaneous port within the dilated opening in the pericardium, wherein the anchor mechanism includes an anchor cuff within the pericardial space, and wherein the anchor mechanism further includes a proximal supra-dermal anchor cuff.

The image guided pericardial access port assembly according to the invention may provide wherein the at least one tracked tool includes at least one of a direct vision tool, an ablation tool, a material delivery tool, a left atrial appendage closure tool, and a lead placement tool.

One aspect of the present invention is directed to a system and a method of performing percutaneous image guided pericardial procedures. The method comprises the steps of: providing a tool tracking system having a display for visualization of virtual representations of tracked tools which is configured to have a field of operation encompassing percutaneous pericardial space access and procedures; providing a patient specific three dimensional model of the patient for use in percutaneous pericardial space procedures that is registered with the tool tracking system for simultaneous display of the model and the virtual representation of the tracked tools on the system display; percutaneously inserting at least one tracked tool for the tool tracking system into the pericardial space; and simultaneously visualizing the virtual representation of the tracked tool and the patient specific model on the system display at least through a portion of a pericardial procedure.

One aspect of the present invention provides a system for performing percutaneous image guided pericardial procedures comprises: a tool tracking system having a display for visualization of virtual representations of tracked tools which is configured to have a field of operation encompassing percutaneous pericardial space access and procedures; a patient specific three dimensional model of the patient for use in percutaneous pericardial space procedures that is registered with the tool tracking system for simultaneous display of the model and the virtual representation of the tracked tools on the system display; and at least one percutaneously inserted tracked tool for the tool tracking system into the pericardial space, wherein the system is configured to simultaneously visualize the virtual representation of the tracked tool and the patient specific model on the system display at least through a portion of a pericardial procedure.

These and other advantages are described in the brief description of the preferred embodiments in which like reference numeral represent like elements throughout.

### BRIEF DESCRIPTION OF THE FIGURES

FIG. 1 is schematic representation of a percutaneous pericardium access tool formed as a needle having a lumen sufficient for emitting dye visible on real time imaging through the lumen and wherein a leading edge of the needle of the percutaneous pericardium access tool is formed to minimize the likelihood of myocardial laceration or puncture in accordance with a first aspect of the invention;

FIG. 2 is schematic representation of the percutaneous pericardium access tool of FIG. 1 with a shaped leading edge;

FIG. 3 is schematic representation of a percutaneous pericardium access tool with a shaped leading edge according to another embodiment of the present invention;

FIG. 4 is schematic representation of a percutaneous pericardium access tool with a shaped leading edge according to another embodiment of the present invention;

FIG. 5 is schematic representation of a five degree of freedom tool tracking coil;

FIG. 6 is schematic representation of a six degree of freedom tool tracking coil;

FIG. 7 is schematic representation of percutaneous pericardium access tool coupled with a tool tracking coil according to another embodiment of the present invention;

FIG. 8 is schematic representation of percutaneous pericardium access tool with RF energy ablation assist coupled with a tool tracking coil via an attachable hub according to another embodiment of the present invention;

FIG. 9 is schematic representation of percutaneous pericardium access tool coupled with a tool tracking coil according to another embodiment of the present invention;

FIG. 10 is schematic representation of percutaneous pericardium access tool coupled with a tool tracking coil according to another embodiment of the present invention;

FIG. 11 is schematic representation of percutaneous pericardium access tool coupled with a tool tracking coil according to another embodiment of the present invention;

FIG. 12 is schematic representation of percutaneous pericardium access tool coupled with a tool tracking coil according to another embodiment of the present invention;

FIG. 13 is schematic representation of percutaneous pericardium access tool coupled with a tool tracking coil according to another embodiment of the present invention;

FIG. 14 is schematic representation of a percutaneous pericardium access tool with a shaped leading edge and with RF energy ablation assist according to another embodiment of the present invention;

FIG. 15 is schematic representation of a percutaneous pericardium access tool with a shaped leading edge and with RF energy ablation assist according to another embodiment of the present invention;

FIG. 16 is schematic representation of a percutaneous pericardium access tool with a shaped leading edge and with RF energy ablation assist according to another embodiment of the present invention;

FIG. 17 is schematic representation of a percutaneous pericardium access tool with a shaped leading edge and with RF energy ablation assist according to another embodiment of the present invention;

FIG. 18 is schematic representation of a percutaneous pericardium access tool with a shaped leading edge and with RF energy ablation assist according to another embodiment of the present invention;

FIG. 19 is schematic representation of a percutaneous pericardium access tool with a shaped leading edge and with RF energy ablation assist according to another embodiment of the present invention;

FIG. 20 is schematic representation of a percutaneous pericardium access tool with a shaped leading edge and with RF energy ablation assist according to another embodiment of the present invention;

FIG. 21-22 are schematic illustrations of the method of accessing the pericardial space of a patient using a patient model and tool tracked access needle according to the present invention;

FIG. 23 is a schematic illustration of a percutaneous port for accessing the pericardial space of a patient using a patient model and tracked pericardial space tools according to the present invention;

FIG. 24 are schematic images of a tracked direct vision tool for image guided pericardioscopy used with the port according to the present invention;

FIGS. 25 and 26 are perspective schematic bottom and top views of a tool tracked ablation tool that can be used with the port according to the present invention;

FIGS. 27 and 28 are schematic perspective views of tool tracked material delivery tools that can be used with the port according to the present invention;

FIG. 29 schematically illustrates an image guided tissue grasping and manipulation tool for use with the port of the present invention; and

FIG. 30 schematically illustrates an image guided lead placement tool for use with the port of the present invention.

### DESCRIPTION OF THE PREFERRED EMBODIMENTS

The inventors have developed an innovative imaging and tool tracking system 100 enabling tools 200 to facilitate percutaneous access to the epicardial surface via the subxiphoid (subX) route and perform incision-less, epicardium-based interventions. Reliable access to the epicardial surface encourages interventional cardiologists (IC) and electrophysiologists (EP) to pursue existing indications, such as ventricular tachycardia (VT) ablation, as well as new opportunities, such as left atrial appendage (LAA) closure and biomaterials introduction. Interest in the epicardium is growing among IC/EP given an accumulating number of patients with implantable cardioverter-defibrillators. A significant minority of these patients will have VT requiring ablation of targets that cannot be reached endocardially as well as the need for LAA closure.

### Shaped Pericardial Perforation Needle Design

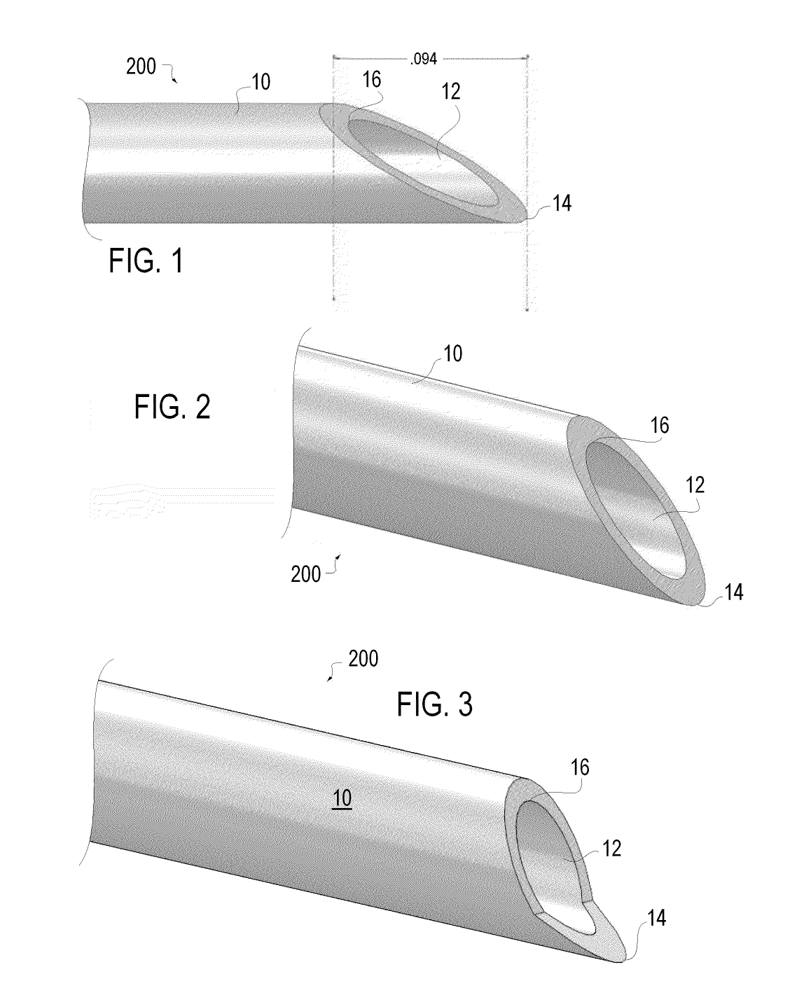

One aspect of the present invention provides an apparatus for accessing the pericardial space including a percutaneous pericardium access tool 10 (one of the class of tools 200 used with system 100 of the invention) having a lumen 12 sufficient for emitting dye visible on real time imaging through the lumen 12 and, as shown in FIGS. 1 and 2, wherein a leading edge 14 of the needle of the percutaneous pericardium access tool 10 is formed to minimize the likelihood of myocardial laceration or puncture.

FIG. 1 illustrates a tool 200 to facilitate percutaneous access in the form of a shaped pericardial perforation needle (or tool) 10 according to a first embodiment of the present invention. The leading end or edge 14 of the needle 10 is moved closer (relative to prior art needles) to the trailing end 16 (top end in the figures) of the lumen 12 to minimize potential myocardial laceration or puncture. The distance from leading end 14 of the needle 10 in FIG. 1 to the midpoint between the trailing end 16 of the lumen 12 and the intersection of the bevel face and the outer diameter is about 0.094″, and this needle is based upon a 20 Gauge Pajunk type needle. A 20 Gauge Pajunk type needle generally exhibits a 0.036 in OD×0.024 in ID. A 0.024 in ID (lumen diameter) needle seemed sufficient for “dye puffs” as well as for a 015 or 020 gauge guidewire passage. The material forming the shaped pericardial perforation needle 10 according to the present invention is conventional.

FIG. 2 shows a similer but more blunted needle 10 than FIG. 1 also generally exhibiting a 0.036 in OD×0.024 in ID. The needle 10 design of the needles 10 of FIGS. 1 and 2 are analogous to the construction of a “blunted” tip in an epidural needle proposed by Dr. Oral Bascom Crawford (b. 1921) described in 1951, with this blunted tip epidural needle being used in over 650 patients for thoracic surgery. The Crawford epidural needle was a very blunt, bevel generally about 60° if measured from the longitudinal axis of the needle. Utilizing the blunted tip design for the shaped pericardial perforation needle 10 of the present invention will allow the lumen 12 to be fully received within the pericardial space without having the leading end 14 of the tip be advanced into the myocardium.

The blunted needles 10 of FIGS. 1 and 2 are not the sole method of moving the leading edge 14 of the needle closer to the trailing edge 16 of the lumen 12. FIG. 3 illustrates a dual bevel design for tool 10, also generally exhibiting 0.036 in OD×0.024 in ID, in which the leading end 14 of the needle (the bottom in the figures) has a sharper angle for ease of tissue penetration, while the upper bevel portion is blunted (less bevel) to pull the leading end 14 of the needle closer to the trailing edge 16 of the lumen 12.

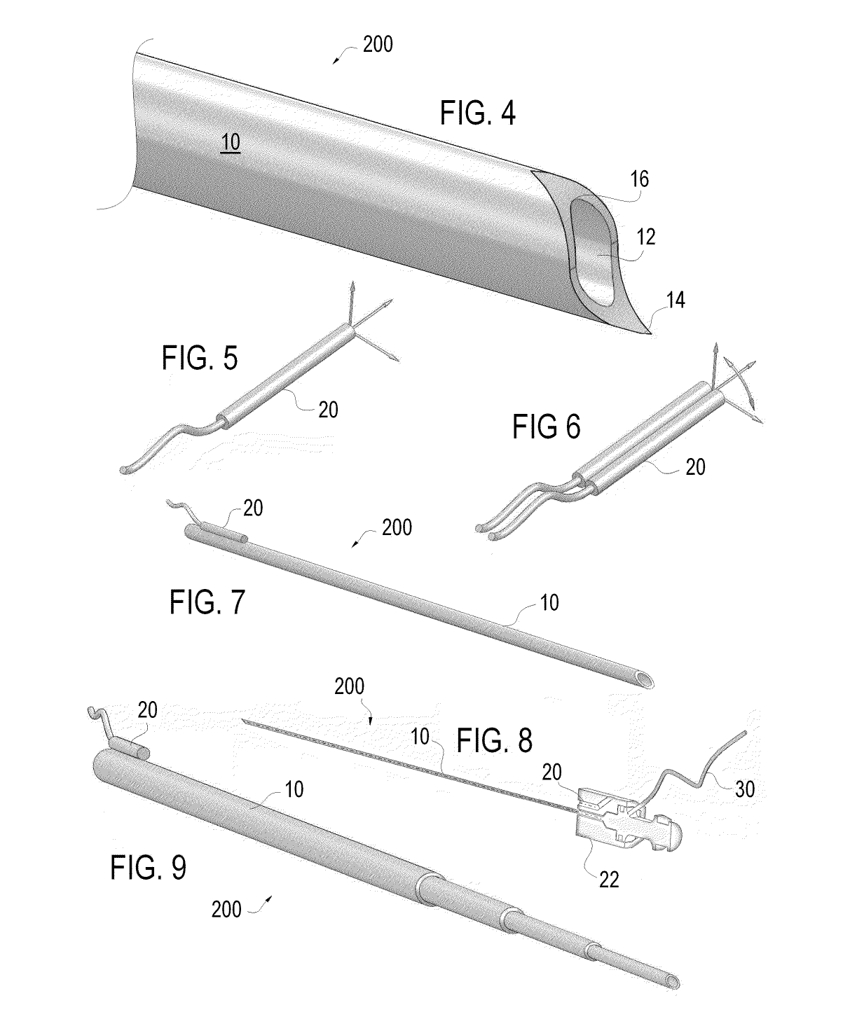

FIG. 4 illustrates an alternative bevel design for tool 10 similer to the dual bevel design of FIG. 3, but is also generally 0.036 in OD×0.024 in ID in shape. In this embodiment as well as FIG. 3, the leading end 14 of the needle (the bottom in the figures) has a sharper angle for ease of tissue penetration, while the midlevel portion is blunted (less bevel) to pull the leading end 14 of the needle closer to the trailing edge 16 of the lumen 12. Here the uppermost portion of the needle 10, generally starting at the trailing end 16 of the lumen 12, begins a sharper bevel, because in the upper portion above the lumen 12 the bevel angle will not affect the position of the trailing edge 16 of the lumen 12 relative to the leading tip 14 of the needle 10 so a sharper angle, as shown may ease the tissue perforation function of the needle 10 in this upper portion. The resulting bevel for tool 10 is an S shaped design as shown in FIG. 4.

As described above in connection with the embodiments of FIGS. 1-4, a “shaped” pericardial perforation needle 10 design according to the present invention will define that the leading end 14 of the needle 10 is moved closer (relative to prior art needles) to the trailing end 16 of the lumen 12 to minimize potential myocardial laceration or puncture. Other configurations are possible in addition to the explicit embodiments of FIGS. 1-4. Additionally other sizes of inner and outer diameters are possible, although the inner and outer dimensions described allow for effective use for pericardial access.

### Tool Tracking

Spatial tracking of surgical tools is readily possible using electromagnetic (EM) sensors. An EM sensor 20 consists of a coiled wire or a set of coiled wires. When a coil 20 is placed in an instrumented magnetic field (formed by field inducing plates or the like), the position and axis orientation (i.e., 5 degrees of freedom) can be determined as illustrated in FIG. 5. The tool tracking system 300 of system 100 will have a display 310 for visualization of virtual representations 320 of tracked tools 200, and in the present application the tool tracking system 300 is configured to have a field of operation encompassing percutaneous pericardial space access.

When a pair of coils 20 are fixed relative to one another (generally with a slight angle relative to the coils) and are placed in an instrumented magnetic field, their position, axis orientation and rotation (6 degrees of freedom) can be determined as illustrated in FIG. 6.

Various strategies can be employed to utilize EM sensors 20 for tracking a surgical pericardial space access needle 10 such as shown in FIGS. 1-4. FIG. 7 is schematic representation of percutaneous pericardium access tool 10, including any of the shaped tips of FIGS. 1-4, coupled with a tool tracking coil 20 according to another embodiment of the present invention. The tool tracking coil 20 is preferably a 6 degree of freedom coil such as represented in FIG. 6. Note that for a five degree of freedom sensor configuration, an axial tracking error exists which is at least equal to the radius of the sensor plus the radius of the needle; a six degree of freedom sensor or a second five degree of freedom sensor is required to rectify this axial tracking error. Thus, two 5 degree of freedom coils may be implemented on opposed sides of the needle to accomplish the same result as a six degree of freedom sensor 20. This rear mounted sensor 20 embodiment for tool 200 requires a certain rigidity in the needle 10 to provide accurate position information as deflection in the needle 10 in use will result in positioning error. However the rear mounted coil attachment allows for easy coupling of a sensor 20 to a needle 10 such as through a hub 22, as shown in FIG. 8. In FIG. 8 a percutaneous pericardium access tool 10, including any of the shaped tips of FIGS. 1-4, is coupled with a tool tracking coil 20 which is mounted in a hub 22 attached to the rear of the needle 10 assembly in a manner not interfering with the perforation function of the needle 10. The hub 22 becomes part of the handle used by the operator/clinician for manipulation of the needle 10.

FIG. 8 also schematically demonstrates the RF energy ablation assist system 30 for percutaneous pericardium access needle 10 according to the present invention which will be discussed in greater detail below. The ablation aspect of system 30 results in an additional electrical cord running from the needle 10 as shown.

FIG. 9 is schematic representation of percutaneous pericardium access tool 10 coupled with a tool tracking coil 20 according to another embodiment of the present invention. The tool tracking coil 20 is coupled to the rear of the needle 10 assembly as shown and is preferably a 6 degree of freedom coil. Alternatively, two 5 degree of freedom coils may be implemented to accomplish the desired result without a positioning error. As noted above, this rear mounted sensor embodiment requires a certain rigidity in the needle 10 to provide accurate position information as deflection in the needle in use will result in positioning error. Addressing this deflection issue and minimizing the undesired deflection, the needle 10 of the percutaneous pericardium access tool of FIG. 9 is formed with a larger proximal diameter than the smaller distal diameter. Specifically three stages of the needle 10 are provided. The larger proximal segments will resist deflection that could yield positioning error in the tracked position. Additionally the larger segments can be used to enlarge an opening in the pericardium once the puncture is properly made, although he tool likely will not be advanced that far.

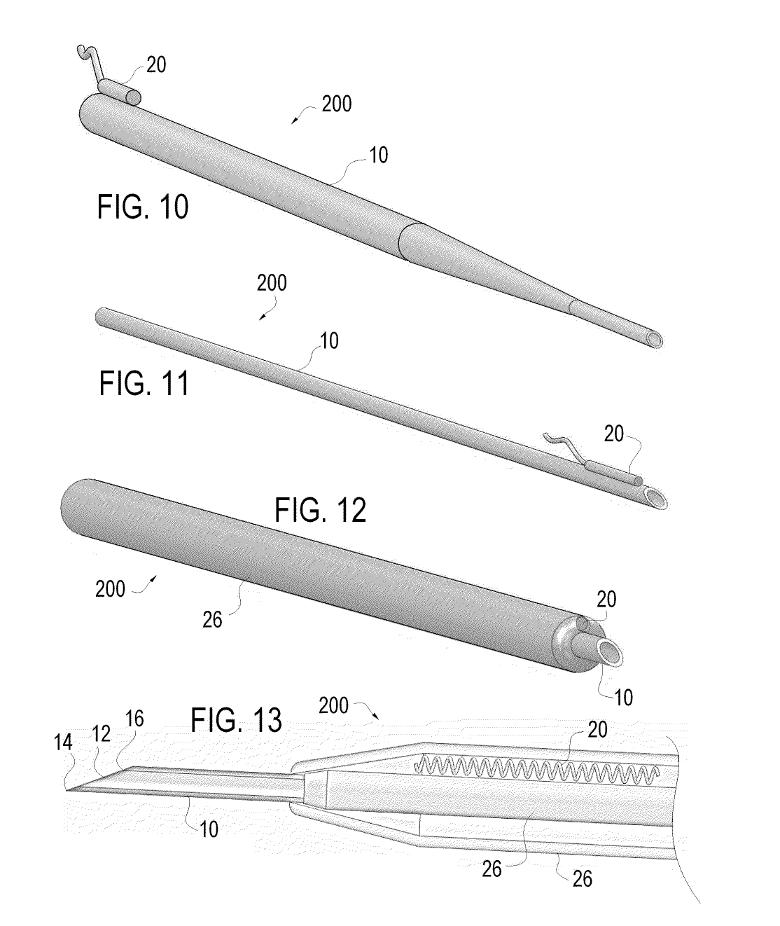

FIG. 10 is schematic representation of a tool 200 in the form of a percutaneous pericardium access tool 10 coupled with a tool tracking coil 20 according to another embodiment of the present invention, similer to FIG. 9. The tool tracking coil 20 is coupled to the rear of the needle 10 assembly as shown. Addressing the aforementioned deflection issue and minimizing the undesired deflection, the needle 10 of the percutaneous pericardium access tool 10 of FIG. 10 is formed with a larger proximal diameter than the smaller distal diameter. Specifically a tapered intermediate segment of the needle 10 is provided. The larger proximal segment and the tapered segment will resist deflection that could yield positioning error in the tracked position. Additionally the tapered segment can be used to enlarge an opening in the pericardium once the puncture is properly made, although he tool likely will not be advanced that far.

FIG. 11 is schematic representation of a tool 200 in the form of a percutaneous pericardium access tool 10 coupled with a tool tracking coil 20 according to another embodiment of the present invention in which the coil 20 is placed close to the piercing tip of the needle 10. The sensor 20 is the same as discussed above. This configuration avoids the problems with needle deflection as it is tracking near the end. The drawback is the potential undesirable interference of the sensor 20 with the handling and operation of the needle 10.

FIG. 12 is schematic representation of a tool 200 in the form of a percutaneous pericardium access tool 10 coupled with a tool tracking coil 20 according to another embodiment of the present invention in which the coil is placed close to the piercing tip of the needle. In this embodiment the needle has a protective sheath 26 around the needle 10 at the distal end that can house the sensor 20. The sensor is the same as discussed above. This configuration avoids the problems with needle deflection as it is tracking near the end, however not all needle configurations have the surrounding sheath adjacent the piecing end and some practitioners may find that this surrounding sheath 26 structure hinders the handling characteristics of the tool 200 and is undesirable from that context.

FIG. 13 is schematic representation of a tool 200 in the form of a percutaneous pericardium access tool 10 coupled with a tool tracking coil 20 according to another embodiment of the present invention. In this embodiment the central needle is based on a 20 Ga Pajunk needle, generally 0.036 in OD×0.024 in ID, as the 0.024 in ID needle seemed sufficient for “dye puffs” as well as 015 or 020 guidewire passage, as noted above. The tool 200 of FIG. 13 uses a trackable needle sheath 26 strategy for coupling the coil(s) 20 to the needle 10. This strategy in the illustrated tool 200 affords a small puncture diameter (via 20 Gauge needle diameter), rigid body manipulation (via 10 Gauge sheath 26 diameter), and enhanced tracking accuracy (via distal sensor 20 placement relative to the tip). The trackable needle sheath 26 may be formed by “sandwiching” at least one EM tracking sensor (NDI) between two concentric sheath members that could be formed from hypodermic tubing elements. For example, a piece of 10 Gauge hypodermic tubing may be modified to make up the outside shell of the trackable needle sheath 26 of the embodiment of FIG. 13, wherein 10 Gauge tubing is generally 0.134 in OD×0.106 in ID. The distal tip of the 10 Gauge tubing for the outer part of sheath 26 is incrementally crimped and polished until the distal ID fit snug over the Pajunk needle OD dimension (0.036 in). A piece of 18 Gauge hypodermic tubing was used to make up the inside shell of the trackable needle sheath 26, wherein 18 Gauge tubing is generally 0.050 in OD×0.038 ID. The EM sensor 20 was attached to the distal tip of the 18 Gauge tubing using thin walled heat shrink tubing and epoxy adhesive. Again a pair of 5 degree of freedom EM sensors may be used to eliminate errors. Following coupling of the EM sensor(s) 20, then the 18 Gauge tubing and sensor 20 was fitted to the distal pocket of the tapered 10 Gauge tubing forming the outer part of sheath 26.

### Tissue Ablation

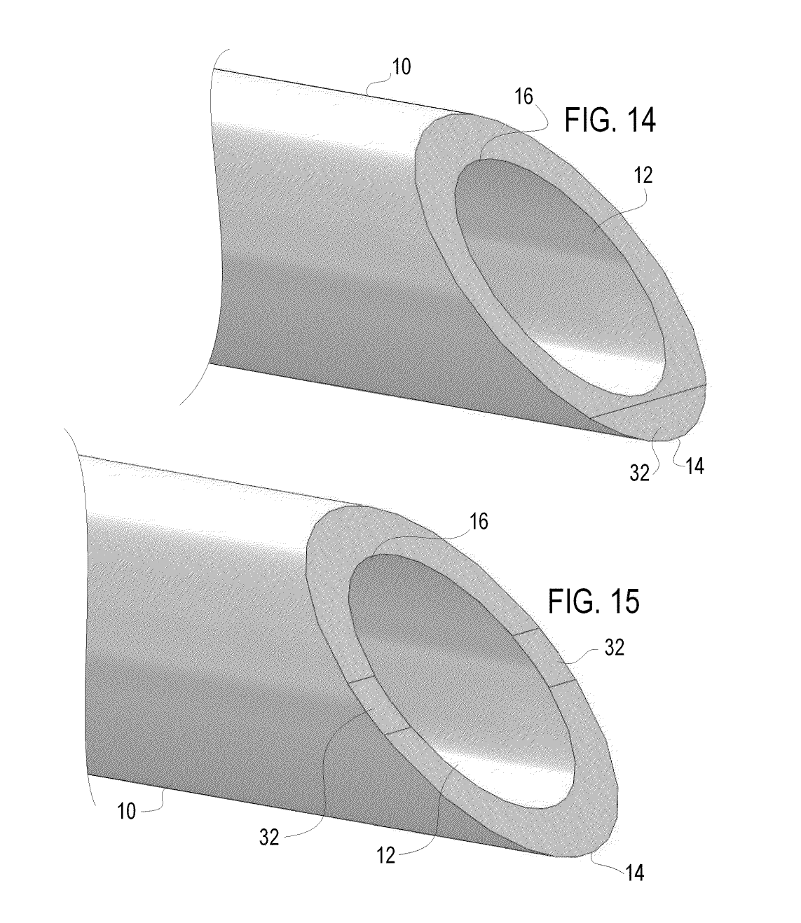

RF tissue ablation technology forming a RF energy ablation assist system 30 might also be integrated within a Pericardial Access System tool 10 or needle assembly for an additional degree of procedural safety. RF energy transmitted through the needle body or through an integrated ablation puncture tool might be used to weaken the tissue adjacent to the pericardial space and reduce the force required to gain access to the pericardial space. This procedural feature might mitigate the likelihood or seriousness of inadvertent myocardial puncture or laceration. An overview of a pericardial access needle 10 with tissue ablation assist system 30 is shown above in FIG. 8. A conductive (metal) needle 10 coated with a non-conductive material on all but selective regions 32 of the distal leading edge from which the RF assist is to emanate is envisioned. Examples are illustrated in FIGS. 14-17. Selective un-coated areas 32 can be used to transmit RF energy to weaken adjacent tissues and reduce the force with which the needle 10 must be advanced in order to puncture/displace tissue while gaining access to the pericardial space.

FIG. 14 is schematic representation of a percutaneous pericardium access tool 10 with a shaped leading edge and with RF energy ablation assist system 30 according to another embodiment of the present invention. Selective un-coated areas 32, namely the bottom/leading edge of the piercing tip, is used to transmit RF energy to weaken adjacent tissues and reduce the force with which the needle 10 must be advanced in order to puncture/displace tissue while gaining access to the pericardial space.

FIG. 15 is schematic representation of a percutaneous pericardium access tool 10 with a shaped leading edge and with RF energy ablation assist system 30 according to another embodiment of the present invention. Selective un-coated areas 32, namely the two segments along the midline of the face of the piercing tip, is used to transmit RF energy to weaken adjacent tissues and reduce the force with which the needle must be advanced in order to puncture/displace tissue while gaining access to the pericardial space.

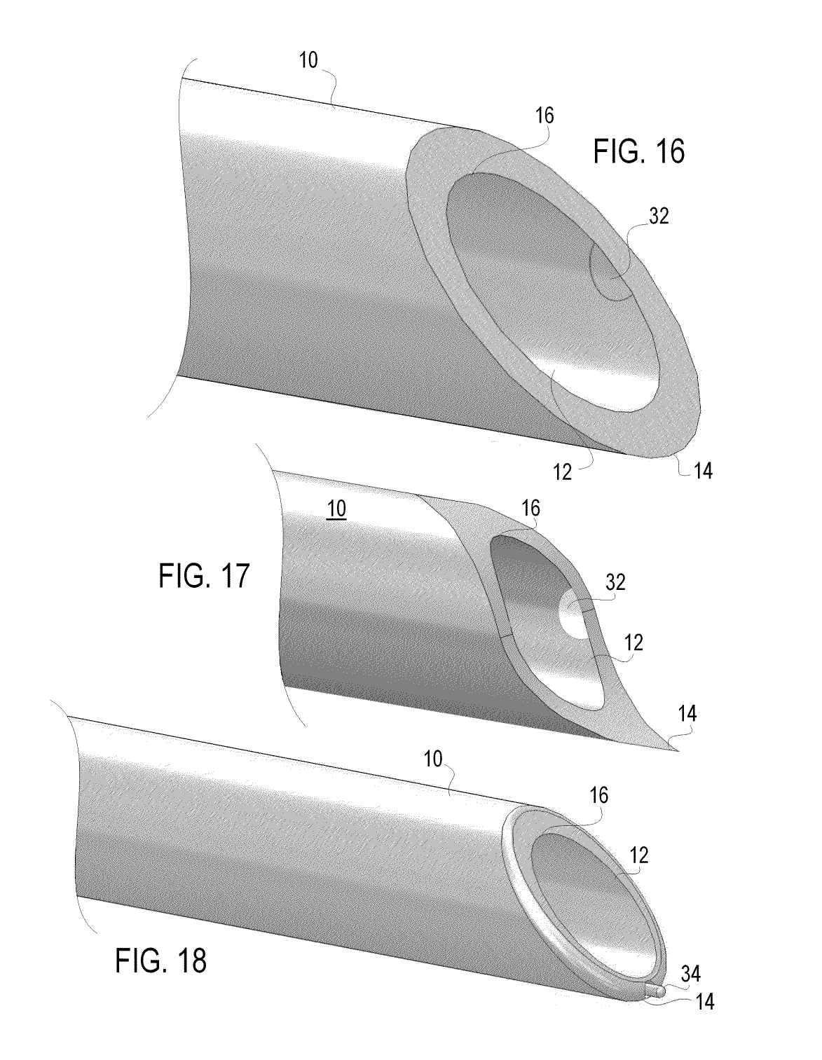

FIG. 16 is schematic representation of a percutaneous pericardium access tool 10 with a shaped leading edge and with RF energy ablation assist system 30 according to another embodiment of the present invention. Selective un-coated areas 32, namely two segments (only one of which is shown) along the midline of the inner diameter and adjacent the piercing tip, is used to transmit RF energy to weaken adjacent tissues and reduce the force with which the needle must be advanced in order to puncture/displace tissue while gaining access to the pericardial space.

FIG. 17 is schematic representation of a percutaneous pericardium access tool 10 with a shaped leading edge and with RF energy ablation assist system 30 according to another embodiment of the present invention. Selective un-coated areas 32, also called ablation pads, namely two segments (only one of which is shown) along the midline of the inner diameter and adjacent the piercing tip, is used to transmit RF energy to weaken adjacent tissues and reduce the force with which the needle 10 must be advanced in order to puncture/displace tissue while gaining access to the pericardial space. FIG. 17 is to illustrate that largely any of the variety of disclosed shaped needle piercing tips can be utilized with any of the variety of ablation pad configurations, and these can be combined with essentially any of the coil coupling concepts disclosed.

FIG. 18 is schematic representation of a percutaneous pericardium access tool 10 with a shaped leading edge and with RF energy ablation assist system 30 according to another embodiment of the present invention. In this embodiment the “ablation pad” is formed from a projection 34 extending from the piercing tip and is used to transmit RF energy to weaken adjacent tissues and reduce the force with which the needle must be advanced in order to puncture/displace tissue while gaining access to the pericardial space.

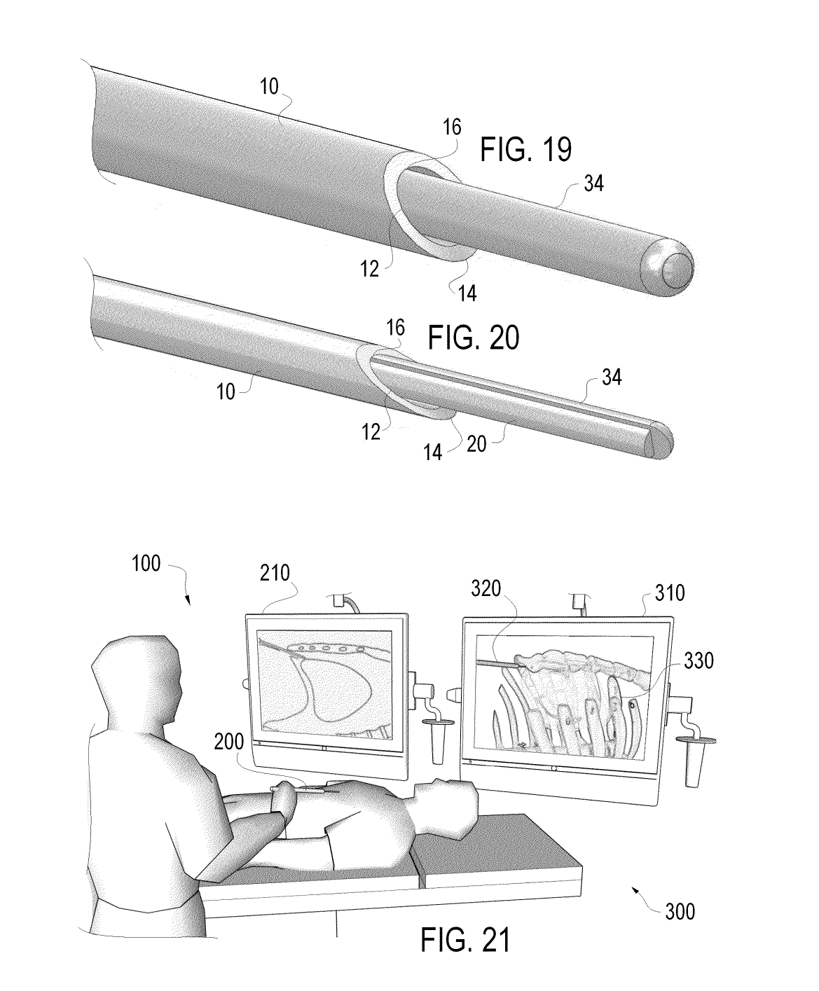

FIG. 19 is schematic representation of a percutaneous pericardium access tool 10 with a shaped leading edge and with RF energy ablation assist system 30 according to another embodiment of the present invention. In this embodiment the “ablation pad” is formed from a separate inner lumen member 34 selectively advanced extending from the piercing tip needle and independent there from, and the projecting pad or member 34 is used to transmit RF energy to weaken adjacent tissues and reduce the force with which the needle 10 must be advanced in order to puncture/displace tissue while gaining access to the pericardial space. This embodiment does allow for independent application of the RF energy assist, but may limit the use of the lumen for dye injection.

FIG. 20 is schematic representation of a percutaneous pericardium access tool 10 with a shaped leading edge and with RF energy ablation assist tool 30 according to another embodiment of the present invention. In this embodiment the “ablation pad” is formed from a separate inner lumen member 34 selectively advanced extending from the piercing tip needle and independent there from generally similer to FIG. 19, and the projecting pad member 34 is used to transmit RF energy to weaken adjacent tissues and reduce the force with which the needle must be advanced in order to puncture/displace tissue while gaining access to the pericardial space. This embodiment additionally couples a EM sensor 20 to the separable RF lead member 34 (by forming the lead member 34 as basically ½ of the lead member 34 of FIG. 19, to provide for tool tracking of the separable RF lead member 34). The RF lead tool tracker shown could be used to supplement the tool tracking results of the tool tracker on the needle 10 as the RF lead member 34 in this embodiment must be extending from the lumen 12.

### Patient Specific Three Dimensional Model of the Patient

One step of the method of accessing the pericardial space of a patient according to the invention includes the step of forming a patient specific three dimensional model 330 of the patient for use in percutaneous pericardial space access. This imaging, in general, is well known in the field, some examples of which are provided by a company known as QuantMD which have developed such models to mine information contained within non-invasive images of the body. For background on modeling techniques see WO-2014-059241 entitled “System and Method for Structure-Function Fusion for Surgical Interventions”, which is publication is incorporated herein by reference. See also U.S. Published Patent Application Nos. 2011-0201915 and 2010-0020926, which are also incorporated herein by reference. Such models have been utilized to assist with decisions of whether a given invasive diagnostic or surgical procedure is appropriate for a prospective patient, to provide personalized planning of the surgical procedure. The proposal here is to use such models to guide the operator during the surgical procedure. QuantMD has developed techniques associated with post-acquisition fusion of anatomically accurate image-derived structural information with custom processed functional evidence depicting morphology, perfusion, function and electrophysiology simultaneously in a single 3D rendering that can be beneficial for such modeling. Other commercial patient modeling systems based upon patient scans are known and may be used.

### Registration

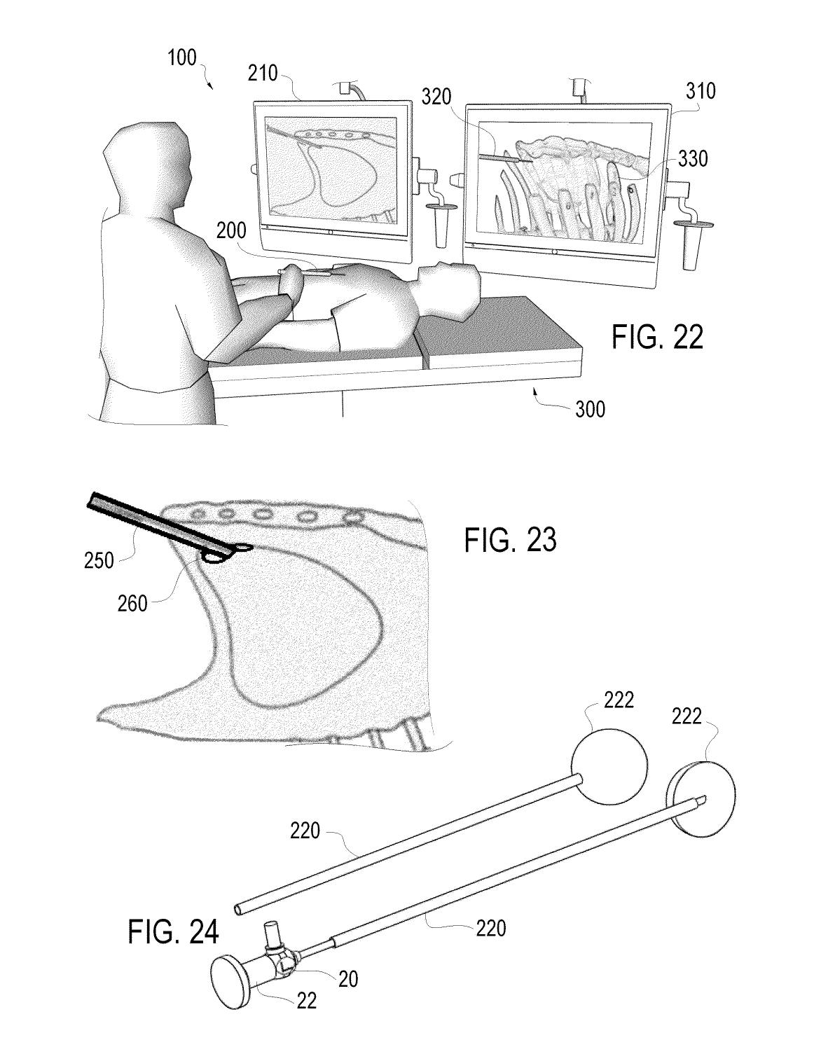

The medical scan of the patient used to form the patient specific three dimensional models 330 is generally one of an MRI and a CT scan. Additionally, the obtaining of a medical scan includes placing scan-able registration markers on the patient, and wherein the patient specific three dimensional model includes representation of the registration markers. The markers are merely fixed indicia that are positioned in fixed locations that will be identifiable in the scan and represented in the model and which will be within the relevant tool tracking field in the operative space for pericardial access when the patient is within the tool tracking field. For example a 3×3 series of metal posts/markers adhesively glued to the patient's skin may easily form the registration markers. The markers will allow for registering of the three dimensional model with the associated tool tracking system display for simultaneous display of the patient specific three dimensional model and the virtual representations of tracked tools on the system display. Essentially for registration of the tool tracking system 300 with the model 330, which can also be called integration of the model 330 into the tool tracking system display 310, the tracked tool 200 is positioned adjacent one or more of the known fixed markers on the patient within the relevant tool tracking field when the patient is not moving within the tool tracking field. In other words, the step of registering the three dimensional model with the tool tracking system 300 includes placing the percutaneous pericardium access tool 10 on a plurality of the registration markers and coordinating the known position of the percutaneous pericardium access tool 10 as determined by the tool tracking system 300 with model 330 position of the associated representation of the registration marker. This allows the model 330 to synchronize with the tool tracking display 310. The synchronization of the model 330 with the tool tracking is schematically shown in the display on the right in FIGS. 21-22, whereas the display 210 on the left illustrates fluoroscopy imaging.

### Real-Time EM-Tracked Tool Position Data

The Real-time EM-tracked tool position data from system 300, when correlated with patient specific models 330 formed from pre-procedural scans of patient anatomy can facilitate vastly improved cardiac access techniques in terms of patient safety. Visual correlation of scanned cardiac anatomy with real-time tool position can provide important feedback to the surgical operator when attempting access to the pericardial space. For example, particular areas of the myocardium might be targeted (adipose tissue) or avoided (superficial vessels) during pericardial access to reduce the risks of bleeding complications in the event of incidental myocardial puncture or laceration.

The method of accessing the pericardial space of a patient according to the present invention may be summarized as comprising the steps of: Forming a patient specific three dimensional model 330 of the patient for use in percutaneous pericardial space access as described above; Configuring a tool tracking system 300 having a display 310 for visualization of virtual representations 320 of tracked tools 200 to have a field of operation encompassing percutaneous pericardial space access as described above; Registering the three dimensional model 330 with the tool tracking system 300 for simultaneous display of the patient specific three dimensional model 330 and the virtual representations 320 of tracked tools 200 on the system display 310; Providing a percutaneous pericardium access tool 200 with a tool tracking sensor 20 for use with the tool tracking system 300 and with a virtual tool representation 320 for the display 310; Simultaneously displaying the virtual tool representation 310 of the percutaneous pericardium access tool 200 and the patient specific three dimensional model 300 at least during movement of the percutaneous pericardium access tool 200 toward the pericardium; and Creating an opening in the pericardium with the percutaneous pericardium access tool 200 (specifically tool 10).

The above described method provides numerous advantages over the prior art access methods, including the tracking of needle 10 and projection of virtual needle geometry via representation 320 over virtual patient anatomy via model 330 created from pre-procedure scans. When used in conjunction with real-time fluorscopy imaging (schematically illustrated on the left in display 210 of FIGS. 21-22), this tracking provides the user an enhanced understanding of the needle 10 trajectory and depth relative to the heart. Further, pre-procedural scan data can detail cardiac landmarks that can be targeted or avoided during pericardial access to minimize risks associated with accidental myocardial puncture (for example, areas surrounding superficial coronary vessels can be avoided, while areas with fatty deposits can be targeted to minimize the risks associated with accidental myocardial puncture).

A separate advantage of the method of the present invention is that custom needle 10 design minimizes potential patient heart damage. For example where the majority of the proximal needle body is rigid and larger diameter while the distal tip of the needle body is small diameter. A small distal needle diameter helps to minimize the risks associated with accidental myocardial puncture, as smaller lacerations of the myocardium are less prone to excessive bleeding. Further a large proximal needle 10 diameter facilitates a more rigid needle body which helps to enhance the accuracy of needle tracking by sensor(s) 20 placed in the hub 22 or sheath 26 of the needle 10. Further the needle tip design described above, the shaped needle 10, is such that the needle 10 is more blunt and has less of a leading edge 14 in front of the needle lumen 12. Real-time fluoroscopy imaging of the radiopaque “dye puffs” is a primary indicator of needle 10 placement in the pericardial space, however, a very “sharp” needle necessitates that a the leading tip of the needle is further in front of the needle lumen where the radiopaque dye is perfused from, whereas a more blunt needle 10 allows the needle lumen 12 to be closer to the leading edge 14, thus minimizing the likelihood of myocardial laceration or puncture while “dye puffs” in the pericardial space is being confirmed.

Another advantage of the present system and method is the RF assist system 30 through the provision of an electrically insulated needle 10 body with selectively placed conduction site(s) 32 at the needle tip and an electrically conductive lead connection in the needle hub such that RF power can be transmitted to the needle tip from an RF generator. Such an ablation needle tip can allow for a less traumatic entry into the pericardial space than traditional needle puncture, requiring less force and perhaps minimizing the likelihood of myocardial puncture or laceration.

The apparatus for accessing the pericardial space according to the present invention may further include a guide, such as a guidewire, configured to be inserted into the pericardial space through a pericardium opening formed by the percutaneous pericardium access tool 10. The method of accessing the pericardial space according to the invention provides the advantage mentioned above of allowing the patient specific model 330 to detail cardiac landmarks utilized during pericardial access to minimize risks associated with accidental myocardial puncture. The cardiac landmarks utilized during pericardial access to minimize risks associated with accidental myocardial puncture may include areas with fatty deposits, and wherein these fatty areas are targeted as general areas to proceed with forming the opening in the pericardium in order to minimize the risks associated with accidental myocardial puncture.

The method of accessing the pericardial space according to the invention as described further comprising the step of utilizing real time imaging, such as fluoroscopy in display 210, at least during the creating of the opening in the pericardium with the percutaneous pericardium access tool 10. The method of accessing the pericardial space according to invention may provide that the percutaneous pericardium access tool 10 includes a lumen 12, and further including the step of emitting dye visible on the real time imaging through the lumen 12.

The system 100 of the present invention may be summarized as an apparatus for accessing the pericardial space of a patient comprising: a tool tracking system 300 having a display 310 for visualization of virtual representations 320 of tracked tools 200 which is configured to have a field of operation encompassing percutaneous pericardial space access; a patient specific three dimensional model 330 of the patient for use in percutaneous pericardial space access that is registered with the tool tracking system 300 for simultaneous display of the model 330 and the virtual representation 320 of the tracked tools 200 on the system display 310; and a percutaneous pericardium access tool 10 with a tool tracking sensor 20 for use with the tool tracking system 300 and with a virtual tool representation 320 for the display 310. The apparatus 100 for accessing the pericardial space according to invention may further include a guide, such as a guide wire, configured to be inserted into the pericardial space through a pericardium opening formed by the percutaneous pericardium access tool 10.

As noted above the apparatus 100 of accessing the pericardial space according to invention provides wherein the patient specific three dimensional model 330 is based upon a medical scan of the patient and wherein the medical scan includes placing scan-able registration markers on the patient, and wherein the patient specific three dimensional model 330 includes representation of the registration markers. Further, the patient specific model 330 may detail cardiac landmarks utilized during pericardial access to minimize risks associated with accidental myocardial puncture, and wherein the cardiac landmarks utilized during pericardial access to minimize risks associated with accidental myocardial puncture includes areas with fatty deposits, and wherein these fatty areas are targeted as general areas to proceed with forming the opening in the pericardium in order to minimize the risks associated with accidental myocardial puncture. The apparatus 100 of accessing the pericardial space according invention may provide wherein the percutaneous pericardium access tool 10 includes a lumen 12, and further including emitting dye visible on the real time imaging such as at 210 through the lumen 12.

As discussed above, the apparatus 100 of accessing the pericardial space according to the invention provides that the percutaneous pericardium access tool 10 includes a needle at a distal end thereof, and wherein the needle of the percutaneous pericardium access tool 10 may be formed with a larger proximal diameter than the smaller distal diameter. Further, a leading edge of the needle of the percutaneous pericardium access tool 10 is formed to minimize the likelihood of myocardial laceration or puncture, and a distal end of the percutaneous pericardium access tool includes a radio frequency ablation tool system 30, and wherein the percutaneous pericardium access tool 10 includes a tool tracking sensor 20 for use with a tool tracking system 300 having a virtual tool representation 320 for the display.

### Port

The above described process of accessing the pericardial space of a patient opens the door for numerous pericardial procedures. With the safe access to the pericardial space, there is a need to allow the easy access of other surgical tools 200, preferably, which tools 200 are also tracked with tool tracking. Facilitating this goal is the placement of a percutaneous port 250 into the opening in the pericardium, such as the port 250 shown schematically in FIG. 23. FIG. 23 is a schematic illustration of a percutaneous port 250 for accessing the pericardial space of a patient using a patient model and tracked pericardial space tools according to the present invention. The sequence of installing the port 250 includes the steps of: forming an opening in the pericardium of a patient as discussed above; placing a guide wire to extend through the formed opening in the pericardium and into the pericardial space; dilating the formed opening in the pericardium; placing a percutaneous port 250 into the dilated opening in the pericardium, wherein the port is configured to receive tools 200 there through into the pericardial space. The percutaneous port 250 may have an anchoring mechanism, such as an inflatable cuff 260 at a distal end thereof to maintain the port 250 in proper position.

The dilation of the original port opening may be performed by advancing dilators along the guide wire into the formed opening in the pericardium, alternatively the port 250 may include a structure to facilitate dilation of the opening. The step of deploying an anchor mechanism to maintain the percutaneous port 250 within the dilated opening in the pericardium may be through an inflatable cuff 260 or other anchoring system. Further the anchor mechanism may further include a proximal supra-dermal anchor cuff.

The method of accessing the pericardial space using a port 250 according to the invention may provide wherein the percutaneous port 250 includes multiple distinct lumens and further including the step of insufflating the pericardial space via the percutaneous port.

As noted above, preferably the tools 200 and possibly the port 250 itself includes tool tracking EM sensors 20 thereon together with virtual representations 320 of the tools 200 and port 250 in the tool tracking system 300, whereby the tool tracking system display 310 provides for visualization of virtual representations 320 of tracked tools 200 for simultaneous display of the patient model 330 and the virtual representation 320 of the tracked tools 200 on the system display 310. Thus the system 100 allows for inserting at least one tracked tool 200 for the tool tracking system 300 into the pericardial space through the port 250 and simultaneously visualizing the virtual representation 320 of the tracked tool 200 and the patient specific model 330 on the system display 310.

The method of accessing the pericardial space according to invention using a percutaneous port 250 preferably provides that at least one of the tracked tools 200 inserted into the pericardial space through the port 250 is a direct vision tool 220, wherein the virtual representation 320 of the direct vision tool 220 includes a visible representation of the field of view of the direct vision tool 220. In addition to at least one of a direct vision tool 220, the system and method provides for an ablation tool 230, a material delivery tool 240, a left atrial appendage closure tool 260, a surgical fastener placement tool, and a lead placement tool 270.

### Image Guided Percardioscopy

FIG. 24 are schematic images of one of the tracked tools 200 inserted into the pericardial space through the port 250, namely a direct vision tool 220. The tool 220 is a fiber optic or other direct vision scope and which includes an inflatable distal end 222 than can be useful in providing space for the fiber optic vision. The upper view schematically illustrates the inflated end 222 while the partial section view below shows the inner optic of tool 220 within the inflatable chamber of the end 222. The lower figure shows a pair of EM sensors 20 at right angles to each other mounted at the base or hub 22 for proper tool tracking in the system 300. The tool tracking system 300 includes a representation 320 of the direct vision tool 220 and in the present invention the virtual representation 320 of the direct vision tool 220 may include includes a visible representation of the field of view of the direct vision tool 220 via a cone or sector. The visible representation of the field of view of the direct vision tool 220 in the virtual representation 320 of the direct vision tool 220 can assist the user in visualizing the direction of view.

The Fiber optic imaging tool 220 of FIG. 24 provides a 2.5 mm OD rigid 30° scope for this purpose. The scope is modified from conventional scopes in two ways: First, the scope contains a sensor(s) 20 that permits it to be visualized in virtual space including, importantly, its visual envelope as demonstrated on the registered preoperative (generally computed tomographic (CT)) image. In addition to seeing in real time the physical location of the scope of tool 220, providing the clinician knowledge of “which direction the scope is looking” will be of great utility to IC/EP who are not endoscopists by training or intuition. Secondly the tool 220 includes a disposable, flexible transparent housing or end 222 placed on the distal portion of the scope before insertion. Once in the pericardial space, this housing can be variably inflated as to create space between scope window and the epicardial surface. Given the tight nature of the “dry” pericardial space, creating such space may be essential for effective imaging. This “local” approach is preferable to more global approaches, such as gas or liquid insufflation.

### Image Guided Pericardial Ablation

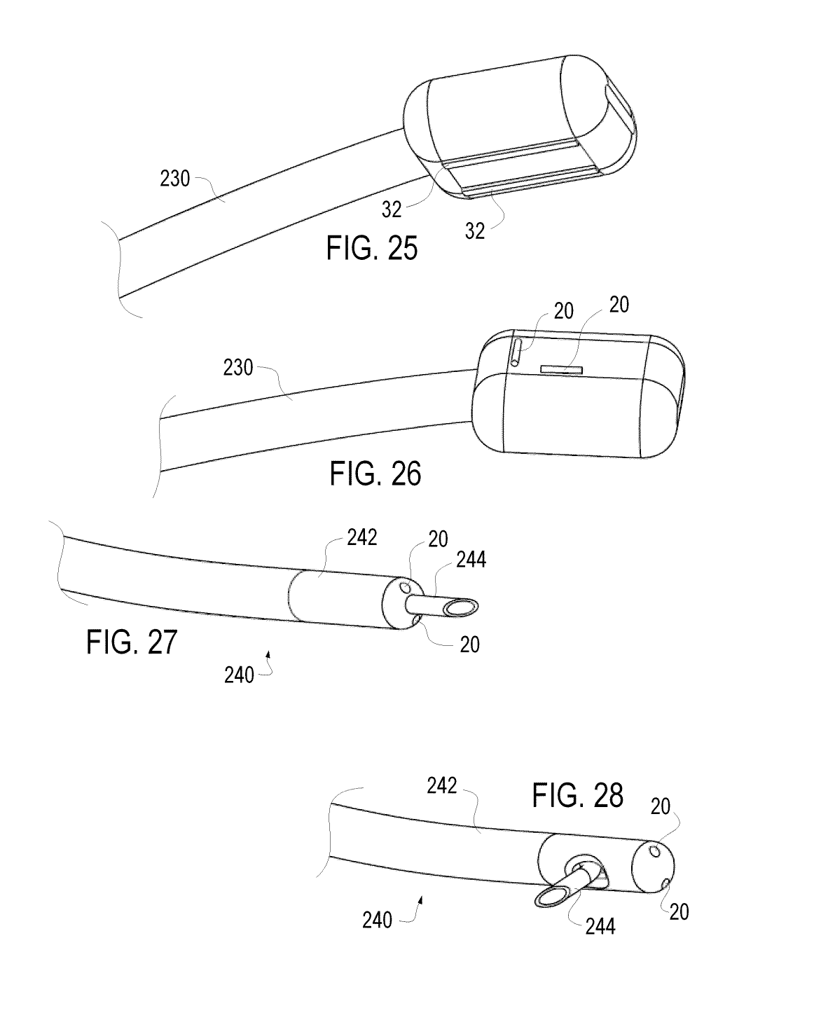

FIGS. 25 and 26 are perspective bottom and top views of a tool tracked ablation tool 230 that can be used with the port of the present invention. The ablation tool 230 includes ablation pads 32 for delivering RF energy to desired tissue and the operation of which is well known in the art. The top view illustrates the placement of a pair of angled 5 degree of freedom coils 20 for tracking of the ablation tool 230 in the system 300 of the present invention. The tool tracking system 300 includes a representation 320 of the image guide ablation tool 230.

### Image Guided Pericardial Material Delivery

FIGS. 27 and 28 are schematic perspective views of tool tracked material delivery tools 240 that can be used with the port 250 according to the present invention. The material delivery tool of FIG. 27 includes an outer lumen 242 carrying a pair of five degree of freedom EM sensors 20 for tool tracking and an inner coaxial, longitudinally extending retractable injection lumen 244. The material delivery tool 240 of FIG. 28 is analogous to the tool 240 of FIG. 27 and includes an outer lumen 242 carrying a pair of five degree of freedom EM sensors 20 for tool tracking and an inner retractable injection lumen 244. In the embodiment of FIG. 28 the injection needle or lumen 244 is positioned to advance radially and may be formed of a shape memory alloy such as Nitinol to easily accommodate a curved advance. The delivery systems or tools 240 of FIGS. 27 and 28 can be inserted through the port 250 into the pericardial space and inject material into tissue at the desired location and/or into the pericardial space. It is possible to have the tools 240 pierce a structure and deliver material within another chamber such as within the interior of the left atrial appendage. The needle or inner retractable lumen 244 length and orientation may be selected according to the desired operation. The tool tracking system 300 includes a representation 320 of each of the material transfer tools 240 of FIGS. 27-28.

### Image Guided Pericardial Tissue Grasping and Manipulation

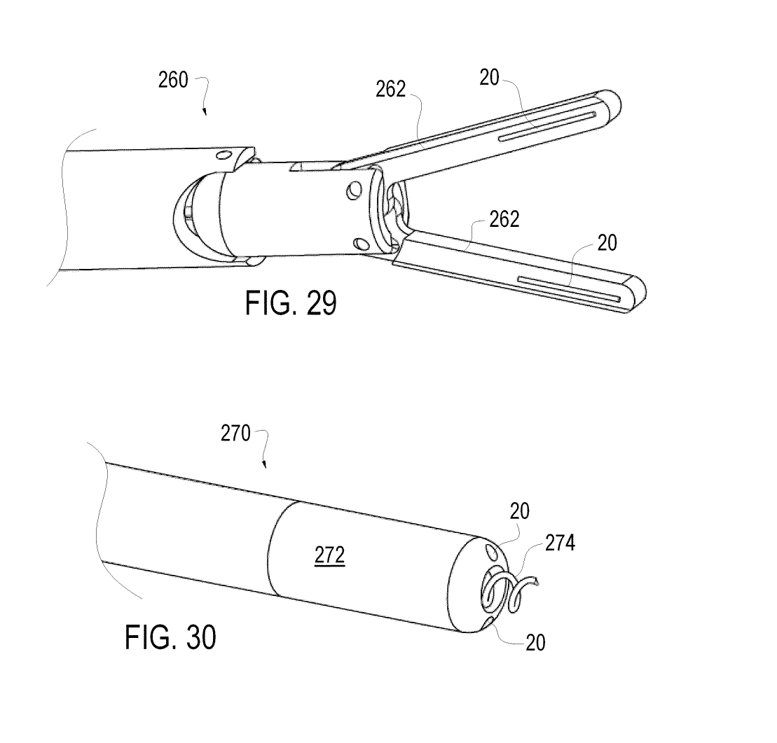

FIG. 29 schematically illustrates an image guided tissue grasping and manipulation tool 260 for use with the port 250 of the present invention. FIG. 29 illustrates two pivoted grasping jaws 262 on the distal end of the tool 260 and which the tool 260 is positioned for rotating the grasping end for proper orientation. The pair of sensors 20 are mounted on the jaws to place them close to the distal end. This tool may be particularly useful for epicardial LAA closure procedures. Each jaw 262 may have a 6 degree of freedom sensor 20 (or pair of 5 degree of freedom sensors) so that each jaw position is accurately shown relative to the other in the virtual representation 320 thereof.

### Image Guided Pericardial Lead Placement

FIG. 30 schematically illustrates an image guided lead placement tool 270 for use with the port 250 of the present invention. FIG. 30 illustrates the lead placement tool 270 as including an outer lumen 272 carrying a pair of five degree of freedom EM sensors 20 for tool tracking and inner longitudinally extending leads 274. The tool tracking system 300 includes a representation 320 of lead placement tool 270 of FIG. 30.

### Image Guided Pericardial Surgical Fastener Placement

The present invention contemplates numerous other image guided tools 200 used with the port 250 including surgical fastener (e.g. surgical tacks) placement tool, essentially similer to the lead placement of FIG. 30.

### System

The present systems 100 includes direct (fiber optic, fluoroscopy via display 210) image guided and synthetic or virtual (image guidance with magnetic tool tracking via display 310) imaging elements. The system 100 provides the clinician with real time vision of surgical tools 200 after they pierce the skin. This approach minimizes “mind's eye work” which is a major hurdle to broad adoption of epicardium-based therapies. This is particularly true for the majority of IC/EP who do not practice in quaternary medical centers. Currently, the system 100 is coupled with tools for pericardial access and port stabilization. Tools 200 specifically configured for use within this environment have been conceptualized for a number of procedures, including ablation, LAA closure, pacing lead placement, and biomaterials introduction.

This technology permits safe, reliable and strategic (anatomically targeted) access, rendering it superior to current “mind's eye” clinical practice. The intense IC/EP interest in pericardial access, coupled with a low capital purchase price point for this technology may promote rapid and broad integration into IC/EP laboratories, and increasing usage as the subX-based procedure portfolio grows. One essential goal of the system 100 is to provide the operator with continuous, real-time, comprehensive visualization of tools 200 after they pierce skin. Although the system 200 may be used in any body region or access route, the system 100 has optimized it for percutaneous, subxiphoid procedures. The system 100 is comprised of hardware and software to support different visualization techniques:

The Virtual tool presentation and sensor-based tool tracking of the system 300 provides the operator with a virtual image 320 as if the operator can see through the chest wall. The system 300 has coupled the tools 200 with a magnetic tracking system as to demonstrate its position in real time with six degrees of freedom. Further, depending on the sensor array 20, tool activity (e.g., opening and closing of jaws 262) is also readily demonstrated.

While the above described individual techniques are available to the disclosed system environment, they are not all co-dependent. This is important because certain procedures will rely more heavily on (and potentially exclude) one or more of the specific techniques. For example, there may be no role for direct fiber optic imaging in the facilitation of pericardial access as described above.

The preferred embodiments described above are illustrative of the present invention and not restrictive hereof. It will be obvious that various changes may be made to the present invention without departing from the spirit and scope of the invention. The precise scope of the present invention is defined by the appended claims and equivalents thereto.

What is claimed is:

1. A method of accessing the pericardial space of a patient comprising the steps of:
a) Forming a patient specific three dimensional model of the patient for use in percutaneous pericardial space access;
b) Configuring a tool tracking system having a display for visualization of virtual representations of tracked tools to have a field of operation encompassing percutaneous pericardial space access;
c) Registering the three dimensional model with the tool tracking system for simultaneous display of the patient specific three dimensional model and the virtual representations of tracked tools on the system display;
d) Providing a percutaneous pericardium access tool with a tool tracking sensor for use with the tool tracking system and with a virtual tool representation for the display;
e) Simultaneously displaying the virtual tool representation of the percutaneous pericardium access tool and the patient specific three dimensional model at least during movement of the percutaneous pericardium access tool toward the pericardium; and
f) Creating an opening in the pericardium with the percutaneous pericardium access tool.

2. The method of accessing the pericardial space according to claim 1 further including the step of inserting a guide into the pericardial space through the formed pericardium opening, wherein the guide is a guidewire.

3. The method of accessing the pericardial space according to claim 1 further including the step of obtaining a medical scan of the patient and wherein the forming the patient specific three dimensional model is based upon the medical scan, wherein the medical scan is one of an MRI and a CT scan, and wherein the obtaining of a medical scan includes placing scan-able registration markers on the patient, and wherein the patient specific three dimensional model includes representation of the registration markers.

4. The method of accessing the pericardial space according to claim 3 wherein the step of registering the three dimensional model with the tool tracking system includes placing the percutaneous pericardium access tool on a plurality of the registration markers and coordinating the known position of the percutaneous pericardium access tool as determined by the tool tracking system with model position of the associated representation of the registration marker.

5. The method of accessing the pericardial space according to claim 1 wherein the patient specific model details cardiac landmarks utilized during pericardial access to minimize risks associated with accidental myocardial puncture.

6. The method of accessing the pericardial space according to claim 5 wherein the cardiac landmarks utilized during pericardial access to minimize risks associated with accidental myocardial puncture include areas with fatty deposits, and wherein these fatty areas are targeted as general areas to proceed with forming the opening in the pericardium in order to minimize the risks associated with accidental myocardial puncture.

7. The method of accessing the pericardial space according to claim 1 wherein the percutaneous pericardium access tool includes a needle at a distal end thereof, wherein a leading edge of the needle of the percutaneous pericardium access tool is formed to minimize the likelihood of myocardial laceration or puncture.

8. An system for performing percutaneous image guided pericardial procedures comprising:
a) a tool tracking system having a display for visualization of virtual representations of tracked tools which is configured to have a field of operation encompassing percutaneous pericardial space access and procedures;
b) a patient specific three dimensional model of the patient for use in percutaneous pericardial space procedures that is registered with the tool tracking system for simultaneous display of the model and the virtual representation of the tracked tools on the system display; and
c) at least one percutaneously inserted tracked tool for the tool tracking system into the pericardial space, wherein the system is configured to simultaneously visualize the virtual representation of the tracked tool and the patient specific model on the system display at least through a portion of a pericardial procedure.

9. The system for of performing percutaneous image guided pericardial procedures according to claim 8 wherein the at least one tracked tool includes at least one of a direct vision tool, an ablation tool, a material delivery tool, a left atrial appendage closure tool, a material grasping tool, a surgical fastener placement tool, and a lead placement tool.

10. The system for performing percutaneous image guided pericardial procedures according to claim 9 wherein the at least one tracked tool includes a direct vision tool providing an image guided pericardioscopy procedure.

11. The system for performing percutaneous image guided pericardial procedures according to claim 10 wherein the virtual representation of the direct vision tool includes a virtual representation of the field of view of the direct vision tool.

12. The system for performing percutaneous image guided pericardial procedures according to claim 9 wherein the at least one tracked tool includes a radio frequency ablation tool performing a pericardial ablation procedure.

13. The system for performing percutaneous image guided pericardial procedures according to claim 9 wherein the at least one tracked tool includes a material delivery tool, wherein the material delivery tool includes a hollow lumen for injection of material.

14. The system for performing percutaneous image guided pericardial procedures according to claim 9 wherein the at least one tracked tool includes a left atrial appendage closure tool.

15. The system for performing percutaneous image guided pericardial procedures according to claim 9 wherein the at least one tracked tool includes a lead placement tool.

16. An image guided pericardial access port assembly for accessing the pericardial space of a patient comprising:
A) a tool tracking system having a display for visualization of virtual representations of tracked tools which is configured to have a field of operation encompassing percutaneous pericardial space access;
B) a patient specific three dimensional model of the patient for use in percutaneous pericardial space access that is registered with the tool tracking system for simultaneous display of the model and the virtual representation of the tracked tools on the system display;
C) a percutaneous port configured to extend through an opening in the pericardium, wherein the port is configured to receive tools there through into the pericardial space; and
D) at least one tracked tool for the tool tracking system configured to be received through the percutaneous port.

17. The image guided pericardial access port assembly according to claim 16 wherein the percutaneous port includes an anchor mechanism to maintain the percutaneous port within the dilated opening in the pericardium.

18. The image guided pericardial access port assembly according to claim 17 wherein the anchor mechanism includes an anchor cuff within the pericardial space.

19. The image guided pericardial access port assembly according to claim 18 wherein the anchor mechanism further includes a proximal supra-dermal anchor cuff.

20. The image guided pericardial access port assembly according to claim 14 wherein the at least one tracked tool includes at least one of a direct vision tool, an ablation tool, a material delivery tool, a left atrial appendage closure tool, a surgical fastener placement tool and a lead placement tool.
